(19) United States
(12) Patent Application Publication
(10) Pub. No.: US 2021/0402338 A1
(43) Pub. Date: Dec. 30, 2021

(54) H2O2 Filter Media Cleaning

(71) Applicant: Darryl Lind, Vero Beach, FL (US)

(72) Inventor: Darryl Lind, Vero Beach, FL (US)

(21) Appl. No.: 17/118,751

(22) Filed: Dec. 11, 2020

Related U.S. Application Data
(60) Provisional application No. 63/043,631, filed on Jun. 24, 2020.

(51) Int. Cl. B01D 46/00; B01D 46/10; B01D 46/44; B08B 3/08; A61L 2/22; A61L 2/26

(52) U.S. Cl. CPC B01D 46/0079; B01D 46/0028; B01D 46/10; B01D 46/444; A61L 2101/02; A61L 2/22; A61L 2/26; B01D 2279/65; B01D 2279/50; B08B 3/08

(57) ABSTRACT

This present application discloses use of a hydrogen peroxide solution for a filter media cleaning and sanitizing system which is designed to dispense the solution to disinfect an air filter. The system is attached to an existing air filter, allowing the system to lightly mist the filter media with the solution. The invention also adjusts the ions in the air system to eradicate germs, bacteria, viruses, and other airborne pathogens, and functions as a sanitization method for any type of air or HVAC system.

### CROSS-REFERENCE TO RELATED APPLICATION

The present application claims priority to, and the benefit of, U.S. Provisional Application No. 63/043,631, which was filed on Jun. 24, 2020 and is incorporated herein by reference in its entirety.

### BACKGROUND

The present invention relates generally to the field of air filter cleaning and sanitizing systems. More specifically, the present invention relates to a Hydrogen Peroxide (H₂O₂) filter media cleaning and sanitizing system which is designed to dispense hydrogen peroxide solution to disinfect an air filter, and is attached to an existing air filter via several U-shaped clips, allowing the system to lightly mist the filter media with the hydrogen peroxide solution. The invention also adjusts the ions in the air system to eradicate germs, bacteria, viruses, and other airborne pathogens, and functions as a sanitization method for any type of air system in homes, commercial applications, businesses, hospitals, care centers, schools, universities, vehicles, trains, aircraft, and more. Accordingly, the present specification makes specific reference to the present invention. However, it is to be appreciated that aspects of the present invention are also equally usable with other like applications, devices and methods of manufacture.

By way of background, air conditioners and similar devices also referred to as HVAC systems (heating, ventilation and air conditioning) are commonly used in homes, commercial applications, hospitals, care centers, schools, universities, businesses, vehicles, trains, aircraft, and more. Conventionally, air conditioners have been equipped with removable filters which are placed behind the air intake vent of the air conditioners to collect particular matter. The filters are an essential part of the air conditioners and are used for the purpose of collecting dust and other pollutants from the air that enters through the vent such that dirt and dust do not become clogged in the ventilation system of the equipment and performance is not degraded.

Usually air inside a room is polluted by various substances such as dust, cigarette smoke, mold, mildew, viruses, germs and other airborne pathogens which may enter the air conditioner system and remain inside the system until the complete system is fully and completely cleaned. In case the air filter is not cleaned for long periods of time, the dirt and dust accumulate on the filter and act as a hinderance to air transmission through the vents and duct work of the air conditioner. These impacts and degrades the performance of the air conditioner system by minimizing air circulation, influencing the cooling or heating effect, reducing air conditioning ability, air flow, etc. and may frequently require servicing of the system. Frequent servicing requirement for the air conditioner system increases the operating costs and out of pocket expense of the unit owner.

Usually, manual cleaning of the air filters is performed by inserting vacuum hoses, brushes and other devices in the duct work in order to remove dirt and dust. The user may remove the air filter attached to the vent of the air conditioning system and may independently clean the filter. However, manual cleaning may not be particularly effective and dirt and dust may remain even after the user cleans the filter. Manual cleaning requires effort and time of the user and may lead to frustration if the air filter remains dirty even after cleaning. Also, proper cleaning of the air conditioner system may require specific cleaning tools, and some users may lack the means to properly disinfect the system. In such scenarios, the user may require an assistance from an expert for cleaning the air conditioner or air transfer system properly, which may add to the budget of the user. An improved air filter cleaning system is desired in the state of the art.

Therefore, there exists a long felt need in the art for a cleaning mechanism for air filters used in air conditioners. There is also a long felt need in the art for an effective and budget friendly cleaning method for air filters. There is also a long felt need in the art for an air filter cleaning mechanism that improves air ventilation and therefore enhances the performance of the air conditioner system. Additionally, there is a long felt need in the art for a cleaning mechanism that is automatic and prevents users from having to repeatedly engage in manual cleaning of the air filters. Moreover, there exists a long felt need in the art for an air filter cleaning mechanism that easily disinfects the air filter and eradicates germs, bacteria, virus, or other airborne pathogens from the air ventilation system. Finally, there is a long felt need in the art for an automatic self-cleaning mechanism for air filters for air conditioners.

The subject matter disclosed and claimed herein, in one embodiment thereof, comprises a hydrogen peroxide (H₂O₂) filter media cleaning and sanitizing system which is designed to dispense hydrogen peroxide solution across the surface of the air filter to disinfect the air filter, and is attached to an existing vent via several U-shaped clips, allowing the system to lightly mist the filter media with a hydrogen peroxide solution. The sanitization system includes a container that has hydrogen peroxide connected to a small meter pump, which is then further connected to plastic tubing placed over the boundary of the air filter. The plastic tubing includes a mist emitter at four or more positions to cover the complete surface area of the air filter. The system in operation, provides a mist supply through the plastic tubing to the mist heads, which further emits a spray of mist or fog over the air filter. The misting or fogging of the solution over the surface serves to eradicates germs, dirt, dust or other harmful pathogens from the air conditioner system. The hydrogen peroxide mist sprayed through the mist heads or emitters adjusts the ions within the system to eradicate pathogens.

In this manner, the hydrogen peroxide (H₂O₂) based air filter cleaning and sanitizing system provides an automated and self-cleaning method for the convenience of the users. The improved air filter cleaning and sanitizing system uses the hydrogen peroxide solution which effectively cleans the air filter and leaves the air conditioner system clean and helps to improve the overall performance of the system as well. The H₂O₂ filter media cleaning and sanitizing system may be marketed as an in-built accessory or may be sold as an after-market product for various devices. Finally, the improved air filter cleaning and sanitizing device is cost-effective and convenient for the users.

### SUMMARY

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one embodiment thereof, includes a hydrogen peroxide (H₂O₂) filter media cleaning and sanitizing system which is designed to dispense hydrogen peroxide solution directly to a filter to disinfect the air filter, and is attached to an existing vent via several U-shaped clips, allowing the system to lightly mist or fog the filter media with hydrogen peroxide solution. The sanitization system has a container that includes hydrogen peroxide connected to a small meter pump, which is then further connected to a plastic tubing placed over the boundary of the air filter. The plastic tubing has a mist emitter at four or more positions to cover the complete surface area of the air filter. The system in operation, provides a mist supply through the plastic tubing to the mist heads, which further emits a spray of mist or creates a fog over the air filter, thereby eradicating germs, dirt, dust or other harmful pathogens from the ventilation system. The hydrogen peroxide mist sprayed through the mist heads or emitters adjusts the ions within the system to eradicate pathogens, germs and the like. The droplet size of the mist or fog is preferably between 1 and 50 microns and more preferably between 10 and 35 microns to permit adequate air time of the mist or fog to penetrate the surface of the air filter.

In an embodiment of the present invention, an air filter cleaning and sanitizing system is disclosed. The air filter cleaning system includes a container having hydrogen peroxide solution, a metered pump, and a pipe connecting the container. The metered pump is connected to plastic tubing that is clipped over the boundary of the air filter. The system includes spray emitters attached to the boundary of the air filter and are connected internally through the plastic tubing.

In a further embodiment of the present invention, the spray emitters are micro spray emitters and dispense micro particles of the hydrogen peroxide solution supplied through the plastic tubing structure of the air filter cleaning system. The cleaning and sanitizing system includes four spray emitters positioned at a mid-point of each of the edges of the boundary of the air filter. The spray emitter has a structure of emitting the spray particles up to a coverage of 180 degrees, and the unique placement of the spray emitters on the boundary of the air filter, covers complete surface area of the air filter.

In an embodiment of the present invention, an air filter cleaning and sanitizing system is disclosed. The air filter cleaning and sanitizing system has a container filled with a hydrogen peroxide solution, a metered pump, a low voltage pump and a toroidal sensor attached to the container. A first pipe connects the container and the metered pump, a second pipe connects the metered pump and a plastic tubing is clipped over the boundary of an air filter, spray emitters are attached to the boundary of the air filter and are connected internally through the plastic tubing.

In yet a further embodiment of the present invention, a method of automatically cleaning and sanitizing an air filter of an air conditioner system is disclosed. The method comprises the steps of initially receiving an input by a toroidal sensor on turning ON of the air conditioner. Then, activating an air filter cleaning and sanitizing system, and supplying a hydrogen peroxide solution to a metered pump through a pipe. The pump meters a specific amount of the supplied hydrogen peroxide solution. A metered amount of the hydrogen peroxide solution is supplied to spray emitters through a plastic tubing placed on the boundary of the air filter. The plastic tubing is connected to the pipe and the hydrogen peroxide is provided through the spray emitters to the surface area of the filter to eradicate germs and other harmful pathogens from the air filter.

In a yet further embodiment of the presently described invention, a combination sanitizing system and air filter is described and includes an air filter having an air flow inlet side, a frame member surrounding the air filter and a surface. A sanitizing system is provided that has a series of mist or fog emitters, with the emitters disposed around a periphery of the frame and for directing a mist or fog onto the surface of the air filter in a direction of air flow from the air flow inlet. The sanitizing system includes a container filled with a sanitizing solution, and the container has a pump and tubing to deliver the sanitizing solution to the emitters. A sensor is provided for activating the sanitizing system and the sensor triggers dispensing of the sanitizing solution upon the air filter receiving air from the air flow inlet side.

In a still further embodiment, a method of sanitizing an air filter is presented and includes the steps of initially providing an air filter having a surface, frame and a perimeter surrounding the frame. Then arranging a series of spray emitters around the perimeter of the frame to provide coverage of the surface. Next, a container is filled with a sanitizing solution and when the system senses movement of air to the air filter a portion of the sanitizing solution is dispensed by a pump to the spray emitters which create a mist or fog over the surface of the air filter to sanitize and clean the surface of the air filter.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of an H₂O₂ filter cleaning system of the present invention in accordance with the disclosed specification;

FIG. 2 provides a close-up view of the air filter of the present invention in accordance with the disclosed description;

FIG. 3 shows a perspective view of spray emitters connected to the air filter and enabling the cleaning mechanism of the present invention;

FIG. 4 illustrates a diagrammatic representation of one possible method of using the H₂O₂ filter cleaning system of the present invention in accordance with the disclosed specification;

FIG. 5 provides a diagrammatic representation of the area of overlap created by the misters of the present invention; and

FIG. 6 shows the direction of airflow interacting with the mist or fog of the present invention.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

As noted above, there exists a long felt need in the art for a cleaning and sanitizing mechanism for air filters of air conditioners. There is also a long felt need in the art for an effective and budget friendly cleaning method for air filters. There is also a long felt need in the art for an air filter cleaning and sanitizing mechanism that improves the air ventilation and therefore enhances the performance of the air conditioner systems. Additionally, there is a long felt need in the art for a cleaning mechanism that is automatic and prevent users from having to engage in repeated manual cleaning of the air filters. Moreover, there exists a long felt need in the art for an air filter cleaning mechanism that easily disinfects the air filter and eradicates germs, bacteria, virus, or other airborne pathogens from the air ventilation system. Finally, there is a long felt need in the art for an automatic self-cleaning mechanism for air filters for air conditioners.

The innovative product of the present invention features a hydrogen peroxide (H₂O₂) filter media cleaning and sanitizing system which is designed to dispense hydrogen peroxide solution to disinfect an air filter, and is attached to and may be retrofitted on an existing vent via several U-shaped clips, allowing the system to lightly mist or fog the filter media with hydrogen peroxide solution. The sanitization system has a container filled with hydrogen peroxide and which is connected to a small meter pump. The meter pump is connected to a plastic tubing that is placed over the boundary of the air filter. The plastic tubing includes a mist or fog emitter at four or more positions to cover the complete surface area of the air filter. The system in operation, provides a mist supply through the plastic tubing to the mist heads, which further emits a spray of mist over the air filter, to help eradicates germs, dirt, dust or other harmful pathogens from the air conditioner or ventilation system. The hydrogen peroxide mist sprayed through the mist heads or emitters adjusts the ions within the system to eradicate pathogens, germs and the like.

The hydrogen peroxide solution may vary depending on the application and use of the solution. For example, 3 to 6 percent is used for household applications, and 10 to 35 percent hydrogen peroxide may be used in certain food-based applications. Solutions of up to 90 percent hydrogen peroxide may be used for industrial situations. Hydrogen peroxide works by creating an effervescent action on the surface which is being treated with the solution by releasing oxygen to clean the area where it is used. A 3% solution with 97% water has been shown to be effective against certain viruses such as those that cause the common cold. In a preferred application, a hydrogen peroxide solution ranging from 3 to about 10 percent is used. The solution may also contain fragrance additives, water or may also be mixed with sodium bicarbonate in order to enhance the oxidizing effect of the hydrogen peroxide.

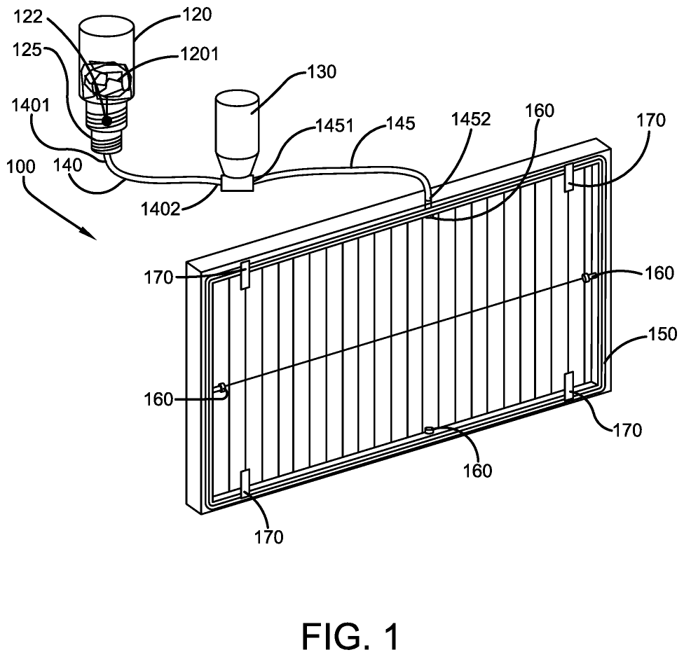

Referring initially to the drawings, FIG. 1 illustrates a perspective view of one possible embodiment of an H₂O₂ filter cleaning and sanitizing system of the present invention in accordance. The hydrogen peroxide air filter cleaning and sanitizing system 100 comprises a container 120 filled with hydrogen peroxide solution 1201, a toroidal sensor 122 attached to the body of the container 120, a low voltage pump 125 attached to a lower end or the dispensing end of the container 125, a metered pump 130, a first pipe 140, a second pipe 145, a plastic tubing structure 150, spray emitters 160 and U-shaped clips 170. The first pipe 140 is a tubular shaped structure of a predefined length, having a first end 1401 and a second end 1402. The first end 1401 is connected to the container 120 having the hydrogen peroxide solution 1201 and the second end 1402 is connected to the metered pump 130. The second pipe 145 is a tubular shaped structure of a predefined length, having a first end 1451 and a second end 1452. The first end is connected to the metered pump 130 and the second end 1452 connects to the plastic tubing structure 150. The plastic tubing structure 150 is configured to be adjustable so that it may be arranged to be in a shape of an air filter 110 and can be easily attached over the boundary of the air filter using U-shaped clips 170. In an embodiment, the plastic tubing structure 150 can be positioned under, over or inside the boundary of the air filter 110.

As shown in the FIG. 1, the air filter 110 is a rectangular shaped structure, and accordingly the plastic tubing structure 150 is also configured to be rectangular in shape. The plastic tubing structure 150 may be flexible to adapt to the configuration of air filters 110 of different shapes. In the preferred embodiment of the present invention, four spray emitter heads 160 are utilized in the automatic air filter cleaning and sanitizing system 100, and each of the spray emitter heads 160 is positioned at approximately a mid-point of each edge of the boundary of the air filter 110. The spray emitter 160 includes a structure of emitting the spray particles up to a coverage of 180 degrees, and the unique placement of the spray emitters 160 on the boundary of the air filter 110, covers the complete surface area of the air filter, and therefore effectively cleans and sanitizes the complete air filter 110. The spray emitter 160 lightly mists or fogs the filter surface with hydrogen peroxide solution 1201 and absorbs and eradicates dust, dirt, germs or other harmful pathogens on the surface of the air filter 110.

The first pipe 140, the second pipe 145 and the tubing 150 is preferably made up of plastic. However, the material of pipe 140, 145 and 150 is not limited and can be made of any other flexible or metallic material. The U-shaped clips 170 are utilized for fastening the tubing 150 to the boundary of the air filter 110. The fastening mechanism is not limited and the tubing 150 may be attached to the boundary using screws, nuts and bolts, double sided tape, adhesives, hook and loop style fasteners or the like.

When in operation, the first pipe 140 supplies the hydrogen peroxide solution 1201 from the container 120 to the metered pump 130, and the second pipe 145 supplies the hydrogen peroxide solution 1201 in a specific metered quantity from the metered pump 130 top to the the tubing structure 150 attached to the boundary of the air filter 110. The tubing 150 supplies the received hydrogen peroxide solution 1201 to the spray emitters 160, which further emits the hydrogen peroxide mist or fog over the surface of the air filter 110 in order to disinfect and sanitize the air filter 110 of the air conditioner or ventilation system.

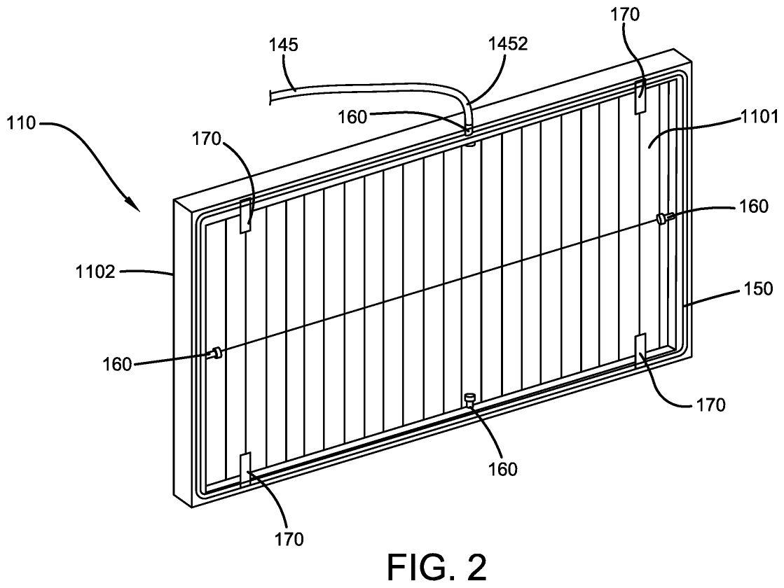

FIG. 2 illustrates a close-up view of the air filter of the present invention in accordance with the disclosed architecture. The air filter 110 has a filtering screen or mesh 1101 surrounded by a solid boundary 1102. The air filter 110 as shown in FIG. 2, is a rectangular shaped structure, and other shapes such as square, rhombus, trapezium, diamond, circular, or the like can be used in the air conditioners as per the needs and desires of the users and the structure of the air conditioner structure. The plastic tubing 150 is a flexible tubular structure which fits to the size and shape of the air filter 110 and is attached over the solid boundary 1102 of the air filter 110. The plastic tubing 150 may be attached under or inside the boundary 1102 of the air filter 110 as well. The plastic tubing 150 is attached to the boundary 1102 of the air filter using four U-shaped clips 170 or other fasteners. The number of U-shaped clips 170 used for fastening the tubing 150 to the boundary 1102 is not limited and any number of the clips 170 may be used to firmly attach the tubing 150 to the boundary 1102. Further, the fastening mechanism is not limited and the tubing 150 may be attached to the boundary 1102 using screws, nuts and bolts, double sided tape, adhesives, hook and loop fasteners or the like.

The plastic tubing internally connects the four spray emitters 160 attached on the mid-point of the edges of the rectangular shaped tubing structure 150. The plastic tubing 150 is connected to the second pipe 145 at the second end 1452 and the hydrogen peroxide solution 1201 is supplied to the tubing 150 and the spray emitters 160 through the second pipe 145. The spray emitters 160 emits the supplied hydrogen peroxide solution 1201 mist or fog with coverage of 180 degrees over the filtering screen or mesh 1101 and spreads the solution 1201 on the complete surface area 1101 of the air filter 110. The sprayed mist adjusts the ions within the system to eradicate the pathogens. Also, the placement of the spray emitters 160 effectively cleans the air filter and improves the overall performance of the air conditioners.

Further, the spray emitters 160 is not limited to cover only up to 180 degrees and may be of different types having different range. Also, other types of mist or fog emitters 160 may be used in the present invention, without deviating and affecting the cleaning and sanitizing mechanism of the present invention.

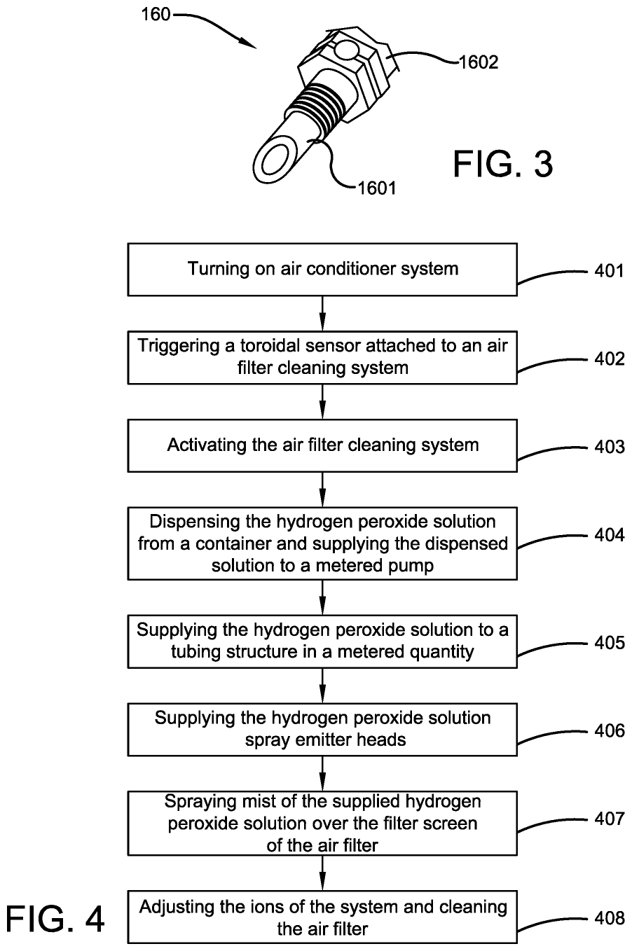

FIG. 3 illustrates a perspective view of spray emitters connected to the air filter and enabling the cleaning and sanitizing mechanism of the present invention. The spray or fog emitter 160 is a screw shaped structure having an opening 1601 which is plugged into the tubing 150 and placed over the boundary or outer perimeter of the air filter 110. The spray emitter 160 has a spray head 1602 with a structure having protrusions at 180-degree angle, in order to spray the mist up to 180 degrees over the surface without wasting the sprayed solution. Other designs of the spray emitters 160 may also be utilized.

FIG. 4 illustrates a diagrammatic representation of one possible method of using the H₂O₂ filter cleaning and sanitizing system of the present invention. The method begins at step 401, wherein an air conditioner or ventilation system is turned ON. At step 402, a toroidal sensor 122 attached to an air filter cleaning and sanitizing system 100 is triggered based on the activation of the air conditioner or ventilation system and initiates an activation of the air filter cleaning and sanitizing system 100. At step 403, the air filter cleaning and sanitizing system 100 is activated based on the input of toroidal sensor 122. At step 404, the hydrogen peroxide solution 1201 is dispensed from a container 1250 and the dispensed solution is supplied to a metered pump 130. At step 405, the hydrogen peroxide solution 1201 is supplied to a tubing structure 150 in a metered quantity or amount. At step 406, the hydrogen peroxide solution 1201 is supplied to one or more spray emitter heads 160 for spraying mist or fog over the air filter 110. At step 407, mist or fog of the supplied hydrogen peroxide solution 1201 is sprayed over the filter screen surface 1101 of the air filter 110. At step 408, the ions of the system are adjusted by the sprayed mist or fog of the hydrogen peroxide solution 110, thereby effectively cleaning and sanitizing the air filter 110 of the air conditioner.

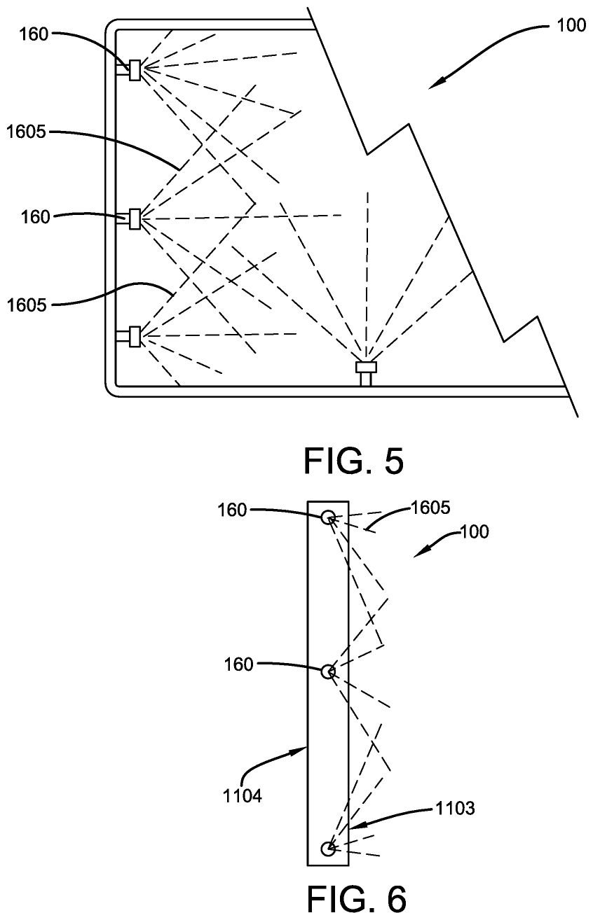

FIG. 5 shows a schematic 500 of the overlapping fields created by the misting pattern of the present invention. Individual misters 510 and 520 have a spray pattern ranging from 0 to 180 degrees. In the present figure, the mister nozzles 510 and 520 are shown with a spray pattern of approximately 90 degrees and are positioned at the corners and mid points of the air filter frame. This then creates an overlapping spray field 515 and 520 of approximately 45 degrees which then ensures complete coverage of the surface area of the air filter. The area of overlapping fields can range from 15 to 90 degrees depending on the angle of dispersion of the nozzles. For example if the nozzles produce a spray pattern of 180 degrees then the area of overlap may be between 45 and 90 degrees or up to half of angle of the spray pattern.

FIG. 6 shows a representation of the air flow direction 700 hitting the mist or fog pattern 710 generated by the misting or fogging nozzles 730. In this way, the air flow contacts the sanitizing solution prior to the air impacting on the filter surface and driving the sanitizing solution on to the surface to clean and sanitize the filter.

In an embodiment, a companion smartphone app is provided to regulate the operation of the air conditioner or the air conditioner can be manually turned ON. The companion smartphone app may also be used to set the metered quantity for the metered pump of the air filter cleaning system 100 of the present invention. The app connects to the device 100 through Wi-Fi, Bluetooth, NFC or other wireless connection technology, where a small wireless connection chip is present in the air filter cleaning system 100. Sensors may be provided on the surface of the air filter which detect the level of contamination of the surface of the filter. The sensors may be used to regulate the flow of sanitizing solution over the surface of the air filter.

The container of the air filter cleaning system 100 may be a reservoir of any shape such as cylindrical or the like, and may be of any size. The container may be designed to hold 100 mL, 200 mL, 300 mL, 400 mL, 500 mL, or the like. The capacity of the container is not limited and can be designed to hold any amount of solution as per the needs and requirements of the user. The container is refillable and can be easily removed from the system to re-fill the solution to enable the operation of the air filter cleaning system 100.

The air filter cleaning system 100 is an automated system which automatically activates as the air conditioner is turned ON. The self-cleaning air filter cleaning system 100 do not require manual operation of the user and automatically cleans the air filter and the air conditioner system. The cleaning system 100 improves the air ventilation and enhances the performance of the air conditioner system. The self-cleaning mechanism 100 for the air filters is a cost-effective method and is convenient to the users. The air filter cleaning system 100 of the present invention may be marketed as an in-built accessory or may be sold as an after-market product for various devices. Further, the air filter cleaning system 100 may be used to eradicate germs, bacteria, viruses, and other airborne pathogens, and functions as a sanitization method for any type of air system in homes, commercial applications, businesses, vehicles, trains, or the like.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “H₂O₂ filter media cleaning”, “hydrogen peroxide air filter cleaning system”, “H₂O₂ filter cleaning system”, “self-cleaning air filter cleaning system” and “automatic air filter cleaning system” are interchangeable and refer to the air filter cleaning system 100 of the present invention.

Notwithstanding the forgoing, the air filter cleaning system 100 of the present invention can be of any suitable size and configuration as is known in the art without affecting the overall concept of the invention, provided that it accomplishes the above stated objectives. One of ordinary skill in the art will appreciate that the size, configuration and material of the air filter cleaning system 100 as shown in the FIGS. are for illustrative purposes only, and that many other sizes of the air filter cleaning system 100 are well within the scope of the present disclosure. Although the dimensions of the air filter cleaning system 100 are important design parameters for user convenience, the air filter cleaning system 100 may be of any size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A cleaning and sanitizing system, comprising;
A container for holding a sanitizing solution, and a sensor connected to the container for controlling the release of the solution from the container;
a first pump connected the container to draw the solution from the container;
a second pump connected to the container and first pump for dispensing a pre-determined quantity of solution;
a series of spray emitters connected to the container and second pump by a plurality of tubes, the tubes feeding the solution to each of the emitters.

2. The cleaning and sanitizing system as recited in claim 1, wherein the spray emitters generate a droplet size of between 1 and 50 microns.

3. The cleaning and sanitizing system as recited in claim 1, wherein the sanitizing solution includes hydrogen peroxide.

4. The cleaning and sanitizing system as recited in claim 3, wherein the amount of hydrogen peroxide ranges from between 3 and 90 percent of the solution.

5. The cleaning and sanitizing system as recited in claim 3, wherein the solution further includes at least one of water, fragrance and sodium bicarbonate.

6. The cleaning and sanitizing system as recited in claim 1, wherein the system includes a series of fastening members to secure the system to a surface for cleaning and sanitizing.

7. The cleaning and sanitizing system as recited in claim 1, wherein the second pump is a metering pump for dispensing pre-determined amounts of solution from the container.

8. The cleaning and sanitizing system as recited in claim 1, wherein the spray emitters produce a mist or fog angle ranging 0 to 180 degrees.

9. The cleaning and sanitizing system as recited in claim 8, wherein the spray emitters produce an area of overlap between spray emitters of up to half of the mist or fog angle.

10. The cleaning and sanitizing system as recited in claim 1, wherein one of the first and second pumps is activated by the sensor after a triggering event.

11. A combination sanitizing system and air filter comprising;
an air filter having an air flow inlet side, a frame member surrounding the air filter and a surface;
a sanitizing system having a series of mist or fog emitters, with the emitters disposed around a periphery of the frame and for directing a mist or fog onto the surface of the air filter in a direction of the air flow inlet;
the sanitizing system including a container filled with a sanitizing solution, the container having a pump and tubing to deliver the sanitizing solution to the emitters; and
a sensor for activating the sanitizing system the sensor triggering dispensing of the sanitizing solution upon the air filter receiving air from the air flow inlet side.

12. The combination as recited in claim 11, wherein the sanitizing solution includes hydrogen peroxide ranging from between 3 and 90 percent of the solution.

13. The combination as recited in claim 11, wherein the sanitizing solution includes at least one of water, fragrance and sodium bicarbonate.

14. The combination as recited in claim 11, wherein the emitters produce a dispersal angle of between 0 and 180 degrees.

15. The combination as recited in claim 11, wherein the emitters produce an overlap pattern of the dispersal angle of up to half of the dispersal angle.

16. The combination as recited in claim 11, wherein the emitters produce a droplet size of between 1 and 50 microns.

17. A method of sanitizing an air filter comprising the steps of;
providing an air filter having a surface, frame and a perimeter surrounding the frame;
arranging a series of spray emitters around the perimeter of the frame to provide coverage of the surface;
filling a container with a sanitizing solution;
sensing movement of air to the air filter;
dispensing a portion of the sanitizing solution by a pump to the spray emitters; and
creating a mist or fog over the surface of the air filter to sanitize and clean the surface of the air filter.

18. The method as recited in claim 17, wherein the emitters produce a droplet size ranging from 1 to 50 microns and dispersal angle ranging from 0 to 180 degrees.

19. The method as recited in claim 18, wherein the emitters produce an overlap pattern of the dispersal angle of up to half of the dispersal angle.

20. The method as recited in claim 17, wherein the sanitizing solution contains between 3 and 90 percent of hydrogen peroxide in solution.
